(19) United States
(12) Patent Application Publication
(10) Pub. No.: US 2024/0288457 A1
(43) Pub. Date: Aug. 29, 2024

(54) SYSTEM INCLUDING ROBOTIC ACTUATOR FOR DIPPING ELECTRICAL SENSOR FOR MEASURING PROPERTIES OF MOLECULES

(71) Applicant: HEXAGONFAB LIMITED, Cambridge (GB)

(72) Inventors: Lukas James VASADI, Cambridge, MA (US); Ruizhi WANG, Cambridge (GB)

(21) Appl. No.: 18/570,991

(22) PCT Filed: Apr. 6, 2022
(86) PCT No.: PCT/EP2022/059117

§ 371 Date: Dec. 15, 2023

(30) Foreign Application Priority Data
Jul. 1, 2021 (DE) 102021117034.6

(51) Int. Cl. G01N 35/00

(52) U.S. Cl. CPC G01N 35/0099; G01N 35/00693; G01N 35/0092; G01N 2035/0097

(57) ABSTRACT

Various examples of the disclosure relate to using a robotic actuator to dip at least one electrical sensor into wells of a multi-well plate.

### TECHNICAL FIELD

Various examples of the disclosure generally relate to measuring properties of molecules. For this, one or more electrical sensors are dipped into liquids including such molecules using a robotic actuator.

### BACKGROUND

Properties of molecules, in particular interactions between molecules, are investigated in various use cases. For instance, for drug discovery and process monitoring, there is a need for low cost, rapid, accurate methods for analysing interactions between molecules.

Examples are numerous and include studying binding kinetics of analyte molecules (e.g., representing future drugs) to target molecules; screening for the quantity and quality of a valuable protein molecule (e.g., monoclonal antibody) during various developmental and production steps; or mapping antigen epitopes to characterise and optimise antibody binding.

These types of measurements are currently performed on a variety of platforms, including High Performance Liquid Chromatography (HPLC) or Enzyme-Linked Immunosorbent Assays (ELISA) and increasingly various label-free assay technologies.

Well-established technologies such as HPLC and ELISA are limited in terms of the range of assays that can be run on these platforms. They also feature comparatively protracted times to result, offer relatively limited data compared to newer technologies and can only be operated by specially trained personnel in a centralised laboratory environment.

The limitations of traditional methods such as HPLC and ELISA have led to a move in recent years to label-free assay platforms. Label-free assays offer various benefits to the user, including real-time data read-out, quantitative data, and high sensitivity. There are several label-free platforms available, mostly based on the optical detection principles of Surface Plasmon Resonance (SPR) and BioLayer Interferometry (BLi). In SPR, sensors made of a thin gold film are used. Incoming light waves are absorbed and excite the electrons in the gold film into a state of collective oscillation, which is referred to as plasmon resonance. BLI is part of the general optical analysis technique called interferometry. A coherent light wave, i.e., light with only one well defined wavelength, is reflected from two surfaces separated by a small distance. The resulting diffraction pattern allows the determination of the distance between the two surfaces. An example of BLi is disclosed in US2011268610 AA.

Optical sensing of molecule properties faces certain restrictions. For instance, the measurement equipment is comparably complex. An optical light path is required to and from a sensor head to a light source and detector of the sensor.

### SUMMARY

Accordingly, there is a need for advanced techniques of measuring properties of molecules. Specifically, there is a need for techniques that facilitate fast, reliable, quantitative, and reproducible measurements.

This need is met by the features of the independent claims. The features of the dependent claims define embodiments.

Hereinafter, techniques of automating measurements for determining properties of molecules of one or more types will be disclosed. Respective measurement protocols defining a sequence of measurement phases and associated actions are disclosed. For instance, binding properties can be between molecules of a first type and molecules of a second type can be determined. Binding properties between target molecules and analyte molecules can be determined.

Such measurements are implemented using an electrical sensor. The electrical sensor may be functionalized. More specifically, a sensitive surface of the electrical sensor may be functionalized using sensor-side molecules.

According to various examples, a robotic actuator is employed to automate the measurements. Specifically, the—possibly functionalized-electrical sensor can be engaged by the robotic actuator and then dipped into one or more wells. The sensor, and a sensitive region of the sensor, can thus be immersed in a liquid. The liquid can be a solution of molecules of a certain type. Molecules could also be included as a dispersion.

By dipping the electrical sensor into liquids including the molecules, it is possible to reliably bring the molecules into contact with a sensitive region of the electrical sensor. Further, a timing of exposure of the sensitive surface to the molecules can be precisely captured based on the dipping process. Complex measurement protocols can be automated, e.g., using a multi-well plate including multiple wells including different liquids. Different phases of a measurement protocol can be implemented. One or more properties can be quantified accurately.

According to various examples, a system includes a robotic actuator. The robotic actuator is configured to engage an electrical sensor. The electrical sensor includes a sensitive surface. The sensitive surface can be functionalized using first molecules of a first type. The system also includes a platform that is configured to retain a multi-well plate (MWP). Also, the system includes at least one processor. The at least one processor is configured to control the robotic actuator to dip the electrical sensor into one or more wells of the MWP. The at least one of the one or more wells are filled with an analyte liquid including second molecules of a second type. The at least one processor is configured to control the electrical sensor to acquire one or more time sequences of data readouts. The at least one processor is further configured to determine at least one property of at least one of the first molecules of the first type and the second molecules of the second time based on at least some of the data readouts of the one or more time sequences of data readouts.

The first molecules can be referred to as sensor-side molecules, because they can be attached to the sensitive surface. The first molecules could also be referred to as ligand molecules. The second molecules can be referred to as liquid-side molecules.

A computer-implemented method includes controlling a robotic actuator. The robotic actuator engages an electrical sensor. The electrical sensor can be functionalized using first molecules of a first type. The robotic actuator is controlled to dip the electrical sensor into one or more wells of a multi-well plate. At least one of the one or more wells is filled with an analyte liquid including second molecules of a second type. The computer-implemented method also includes controlling the electrical sensor to acquire one or more time sequences of data readouts when the electrical sensor is dipped into each one of the one or more wells. The computer-implemented method also includes determining at least one property of at least one of the first molecules or the second molecules based on at least some of the data readouts.

A computer program or a computer program product or a computer-readable storage medium includes program code. The program code can be loaded and executed by at least one processor. Upon loading and executing the program code, the at least one processor performs a method. The method includes controlling a robotic actuator. The robotic actuator engages in electrical sensor. The electrical sensor can be functionalized using first molecules of a first type. The robotic actuator is controlled to dip the electrical sensor into one or more wells of a multi-well plate. At least one of the one or more wells is filled with an analyte liquid including second molecules of a second type. The computer-implemented method also includes controlling the electrical sensor to acquire one or more time sequences of data readouts when the electrical sensor is dipped into each one of the one or more wells. The computer-implemented method also includes determining at least one property of at least one of the first molecules or the second molecules based on at least some of the data readouts.

It is to be understood that the features mentioned above and those yet to be explained below may be used not only in the respective combinations indicated, but also in other combinations or in isolation without departing from the scope of the invention.

### BRIEF DESCRIPTION OF THE DRAWINGS

FIG. 1 schematically illustrates a system for performing measurements on molecules according to various examples.

FIG. 2 schematically illustrates positioning an electrical sensor attached to a robotic actuator with respect to wells of a MWP according to various examples.

FIG. 3 schematically illustrates the electrical sensor being retracted into the well of the MWP according to various examples.

FIG. 4 schematically illustrates the electrical sensor being immersed in a fluid in the well of the MWP according to various examples.

FIG. 5 is a flowchart of a method according to various examples.

FIG. 6 schematically illustrates a time sequence of data readouts according to various examples.

FIG. 7 schematically illustrates a time sequence of data readouts according to various examples.

### DETAILED DESCRIPTION OF EMBODIMENTS

Some examples of the present disclosure generally provide for a plurality of circuits or other electrical devices. All references to the circuits and other electrical devices and the functionality provided by each are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical implementation that is desired. It is recognized that any circuit or other electrical device disclosed herein may include any number of microcontrollers, a graphics processor unit (GPU), integrated circuits, memory devices (e.g., FLASH, random access memory (RAM), read only memory (ROM), electrically programmable read only memory (EPROM), electrically erasable programmable read only memory (EEPROM), or other suitable variants thereof), and software which co-act with one another to perform operation(s) disclosed herein. In addition, any one or more of the electrical devices may be configured to execute a program code that is embodied in a non-transitory computer readable medium programmed to perform any number of the functions as disclosed.

In the following, embodiments of the invention will be described in detail with reference to the accompanying drawings. It is to be understood that the following description of embodiments is not to be taken in a limiting sense. The scope of the invention is not intended to be limited by the embodiments described hereinafter or by the drawings, which are taken to be illustrative only.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Hereinafter, techniques of performing a measurement of one or more properties of one or molecules will be described. Specifically, properties of analyte molecules could be determined.

Such measurements may facilitate various use cases. Surface science can be facilitated where properties of a surface formed by certain molecules is investigated. For instance, proteins or small molecules for research of pharmaceutical drugs may be facilitated. Ribonucleic acid (RNA) or Deoxyribonucleic acid (DNA) can be investigated. Molecule-binding assays could be implemented. Antibody-antigen kinetics could be measured. The quantity and/or quality of a protein molecule such as a monoclonal antibody could be measured. Antigen epitopes could be mapped to characterize and optimize antibody binding.

As a general rule, different kinds of measurements can be implemented and, along with different kinds of measurements, different properties can be determined. For instance, it would be possible to determine a property of a binding/adsorption between target molecules and analyte molecules. Binding parameters may include binding kinetics or a binding affinity. The binding kinetics can specify how fast the target and analyte molecules bind. Binding affinity can specify a strength of the binding. Another option would be to determine a concentration of the analyte molecules in an analyte liquid. Yet another option be to determine a conformality structure of analyte molecules.

Electrical sensing is employed. Specifically, an electrical sensor is used. This means, that an electrical sensor signal is used to determine one or more properties as identified above. For instance, a time-dependency of the electrical sensor signal could be used to determine the binding kinetics.

As a general rule, according to the various examples disclosed herein, various kinds and types of electrical sensors can be used.

Some examples are summarized in TAB. 1.

TABLE 1
Various options for electrical sensors used for measurements.

| | Brief | |
|---|---|---|
| Example | description | Example details |
| I | Field-effect | One example would be a field-effect transistor that |
| | transistor (FET) | includes a sensitive region. Here, electrical fields can |
| | | be created/changed due to presence of molecules at |
| | | or adjacent to the sensitive region. The sensitive |
| | | region thus forms a gate contact of the FET. The |
| | | electrical fields, in turn, can then affect mobility of |
| | | charge carries in a channel between source and drain |
| | | contacts below the gate contact. Thus, a current |
| | | through the FET can be measured and one or more |
| | | properties of molecules can be determined based on |
| | | the current. |
| | | There are, furthermore, various options available to |
| | | implement the sensitive region. |
| | | In some examples, the sensitive region comprises |
| | | graphene as an electrical field sensitive layer. |
| | | Thereby, a material with a large change in electrical |
| | | properties (e.g., resistance) in response to an applied |
| | | electric field can be provided as the electric field |
| | | sensitive layer thus providing a high sensitivity to |
| | | electric fields caused by molecules arranged adjacent |
| | | to the sensitive region. |
| | | In some examples, the sensitive region includes one |
| | | or more of nanowires, nanotubes, and a two- |
| | | dimensional material as an electrical field sensitive |
| | | layer. Materials which can form suitable nanowires |
| | | include, for example, silicon, gallium arsenide (GaAs), |
| | | indium arsenide (InAs) and Galium Nitride (GaN). |
| | | Materials which can form suitable nanotubes include, |
| | | for example, carbon and transition metal |
| | | dichalcogenide. Materials which can form suitable |
| | | two-dimensional materials include, for example, |
| | | graphene, phosphorene, silicene, germanene and |
| | | transition metal dichalcogenides. Thereby, through the |
| | | provision of low dimensional materials, a sensitive |
| | | region which has a high sensitivity to electric fields |
| | | can be provided. |
| | | In some examples, the electric field sensitive layer |
| | | comprises a bulk semiconductor as an electrical field |
| | | sensitive layer. Thereby, inexpensive and readily |
| | | available materials with established processing |
| | | techniques can be used for the electric field sensitive |
| | | layer. Examples of suitable semiconductors include: |
| | | silicon, germanium, gallium arsenide (GaAs), an alloy |
| | | of silicon and germanium, indium phosphide and |
| | | gallium nitride. |
| | | An example implementation of the FET is disclosed in |
| | | WO 2020/099890. The respective disclosure is |
| | | incorporated herein by reference. |
| II | Micro-electrical | A MEMS-based sensor can, e.g., include a |
| | mechanical | mechanical oscillator. The sensitive region could then |
| | system | be implemented by an oscillating mass of the |
| | (MEMS) | mechanical oscillator. Molecules binding to or |
| | | arranged adjacent to the oscillating mass can affect a |
| | | resonance frequency of the oscillating mass. Thus, a |
| | | frequency-shift of the resonance frequency can be |
| | | used as electrical signal to determine one or more |
| | | properties as identified above. |
| III | Electrochemical | An electrochemical cell including a working, a |
| | Sensor | reference or a working, a reference and a counter |
| | | electrode. Molecules binding on the working electrode |
| | | alter the properties of the electrochemical cell. This |
| | | can be read out using a multitude of methods, e.g., |
| | | linear voltammetry, cyclic voltammetry, amperometry, |
| | | potentiometry, electrical impedance spectroscopy |

According to various examples, the sensitive region can be functionalized. According to various examples, it would be possible that the sensitive region is pre-functionalized, i.e., sensor-side molecules are already attached to the sensitive region. It would also be possible that the sensitive region is being functionalized using techniques disclosed herein in a preparation phase of the measurement protocol. In the preparation phase, the electrical sensor is dipped into a liquid of sensor-side molecules. The sensor-side molecules then can adhere to the sensitive region, to thereby functionalize the electrical sensor. To facilitate such binding, the sensitive region can include an adhesion layer to support adhesion of the sensor-side molecules. This could be, e.g., a hexagonal boron nitride layer.

According to various examples, the electrical sensor is dipped into one or more wells, e.g., of a MWP, to bring a sensitive region of the electrical sensor into contact with respective liquid-side molecules included in a respective liquid, e.g., in solution.

Such dipping can be automated using a robotic actuator. The robotic actuator can engage one or more electrical sensors, cf. TAB. 1. Then, an MWP can be arranged on a platform and a processor can be configured to control the robotic actuator to dip the one or more electrical sensors into one or more wells of the MWP.

In particular, it would be possible to use a measurement script. The measurement script can specify different actions of the robotic actuator. Thereby, the measurement protocol can be implemented. For instance, the timing of the dipping, e.g., start time and duration, can be appropriately set. A sequence of wells of the MWP into which an electrical sensor is dipped can be set. Sampling durations during which data readouts are performed can be defined. The electrical sensor can be controlled to acquire one or more time sequences of data readouts in accordance with the predefined measurement script.

Then, based on such data readouts, the at least one processor can be configured to determine at least one property of molecules of one or more types. For instance, a binding property between target molecules and analyte molecules may be measured. It would be possible to measure a property of analyte molecules, e.g., activity, concentration, etc. The analyte molecules can be implemented by the sensor-side molecules or the liquid-side molecules.

Specifically, according to various examples, it would be possible to take into account the measurement script when determining the at least one property. Here, it would be possible that, based on the measurement script, data readouts or time sequences of data readouts are associated with certain liquids to which the electrical sensor is exposed when being dipped into respective wells. Specifically, it would be possible that based on the measurement script, data readouts or time sequences of data readouts are associated with different phases of a measurement protocol, e.g., preparation phase, an association phase, a calibration phase, or a dissociation phase. Thus, it would be possible that the at least one property is determined based on a timing of said controlling of the robotic actuator to dip the electrical sensor into the one or more wells of the MWP. This timing can be defined by the measurement protocol.

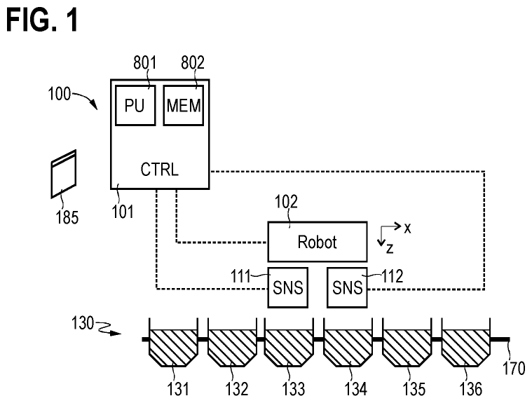

FIG. 1 schematically illustrates a system 100 according to various examples. The system 100 is configured for performing measurements to determine one or more properties of molecules. Specifically, properties of analyte molecules can be determined. The analyte molecules can be in a liquid, i.e., liquid-side molecules; but it would also be possible that the analyte molecules are attached to a sensor surface, i.e., sensor side molecules. An electrical sensor 111 that is used for the measurements can be functionalized using sensor-side molecules.

To determine properties of the analyte molecules, it would be possible to use target molecules. One or more properties of the target molecules may be known. Then, based on prior knowledge, it is possible to determine the one or more properties of the analyte molecules. The target molecules (analyte molecules) can be implemented by the sensor-side surface molecules (liquid-side molecules); or vice versa.

In some experiments, an antibody is immobilized on the sensor (i.e., as analyte molecule) and the target protein is measured in solution. It could also be implemented such that the target protein is immobilized on the sensor and the antibody is in solution as liquid-side molecule.

The system 100 includes a control device 101 that can communicate with a robotic actuator 102 and electrical sensors 111, 112. The electrical sensors 111, 112 are both engaged by the robotic actuator 102. In particular, the electrical sensors 111, 112 could be releasably engaged, so that they can be replaced in between subsequent measurements.

The control device 101 can include at least one processor 801 (labelled “PU” in FIG. 1, processor unit) and a memory 802. The at least one processor 801 could be implemented by a general-purpose processing unit, and an application-specific integrated circuit, or a field-controlled gated array, to give just a few examples. The at least one processor 801 could load program code from the memory 802 and execute the program code. Upon loading and executing the program code, the at least one processor 801 can perform techniques as described herein, e.g., control the robotic actuator to move, e.g., to position the electrical sensors 111, 112 in or above certain wells 131-136 of a MWP 130 that is arranged on a platform 170 or to dip the electrical sensors 111, 112 into respective wells 131-136, determine one or more properties of analyte molecules, read a predefined measurement script 185 and control the robotic actuator 102 and/or the electrical sensor 111, 112 based on the predefined measurement script 185, etc.

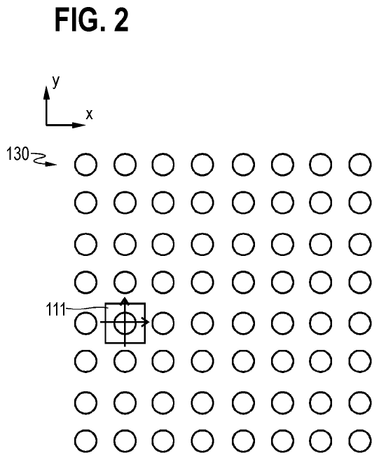

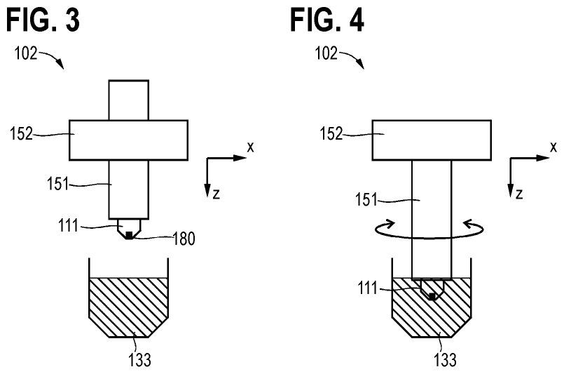

The robotic actuator 102 can be controlled by the control device 101 to move the electrical sensors 111, 112. Depending on the structural implementation of the robotic actuator 102, different degrees of freedom of movement of the robotic actuator 102 are possible. For instance, a translational movement, e.g., along all three spatial axes, would be possible. Here, a lateral movement (along X axis and Y axis) could be used to select a specific well of a multi-well plate (this is illustrated in FIG. 2, where a 2-D array of wells of the MWP 130 is illustrated using the circles), and a depth movement (along the Z axis) could be used to dip the electrical sensor into a respective well 131-136 of the MWP 130 (this is illustrated in FIG. 3 which shows a retracted position and in FIG. 4 which shows a dipped position; here a piston 151 of the robotic actuator 102 moves with respect to a base plate 152, to implement the depth movement). It would also be possible that the robotic actuator 102 can perform a rotational movement, e.g., rotation around the Z axis (cf. FIG. 4). This can be helpful to stir the electrical sensors 111, 112 when dipped in a respective liquid in the wells 131-136.

In the example illustrated in FIG. 1, the robotic actuator 102 could perform a movement along the Z axis; this would result in dipping the electrical sensor 111 into the well 133 and the electrical sensor 112 into the well 134. Thus, because the electrical sensor 111 and the electrical sensor 112 are arranged at an offset with respect to each other (when engaged by the robotic actuator 102) that corresponds to the offset between the wells 131-136 of the MWP, it is possible to jointly dip multiple electrical sensors 111, 112 into separate wells with a single translational movement of the robotic actuator 102.

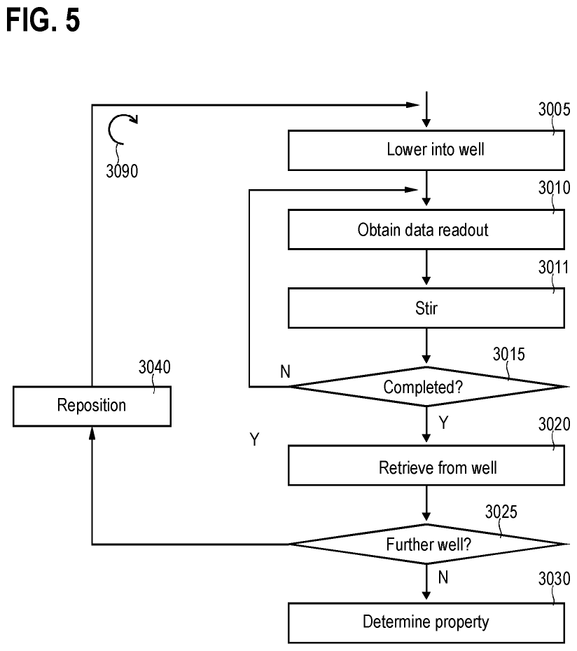

FIG. 5 is a flowchart of a method according the various examples. The method of FIG. 5 could be executed by at least one processor of a measurement system. For instance, the method of FIG. 5 could be executed by at least one processor 801 of the control device 101 of the system 100 of FIG. 1.

At box 3005, an electrical sensor lowered into a well of an MWP. This can include controlling a robotic actuator to lower the electrical sensor into the well (Z-movement). For instance, respective digital control instructions may be provided to the robotic actuator. Also, analog control, e.g., using voltage levels, would be possible, depending on the structural implementation of the robotic actuator.

Then, at box 3010, a data readout or multiple data readouts can be obtained. Box 3010, accordingly, can include controlling the electrical sensor to acquire a data readout. The data readout is representative of an electrical observable sensed by a sensitive region of the electrical sensor when the electrical sensor is immersed in a fluid in the well, e.g., frequency shift for MEMS (cf. TAB. 1, example II) or current for FETs (cf. TAB. 1, example I).

At optional box 3011, it would be possible that—while the sensitive region of the electrical sensor remains immersed in the fluid included in the well into which the electrical sensor has been lowered at box 3055—the robotic actuator is controlled to move the electrical sensor within and with respect to the respective well. For instance, this could include a rotation to stir (cf. FIG. 4) or a shaking movement. Thereby, local concentration gradients of molecules in solution in the respective liquid are avoided.

Alternatively or additionally to controlling the robotic actuator, it would also be possible to control a motor attached to a platform on which the MWP is mounted the electrical sensor to move the platform against the electrical sensor.

At box 3015 it can be checked whether one or more further data readouts are required while the electrical sensor is immersed in a fluid in the well. In the affirmative, further data readouts are obtained by one or more further iterations of box 3010. Thereby, a time sequence of data readouts is obtained, when and while the electrical sensor is dipped into the well.

On the other hand, if it is judged at box 3015 that a sampling duration during which the time sequence of data readouts is obtained by multiple iterations of box 3010 is completed, the method commences at box 3020.

At box 3020, the electrical sensor is retrieved from the well. Box 3020 can include controlling the robotic actuator to retrieve the electrical sensor from the well (Z-movement).

Box 3005 and box 3020 thus implement dipping the electrical sensor into a respective well of the MWP. A time offset between executing box 3005 and box 3020 defines a dwell time of the electrical sensor in the respective well.

At box 3025, it is checked whether the electrical sensor is to be dipped into a further well. In the affirmative, boxes 3005, 3010, 3015, and 3020 are executed in a respective iteration 3090, after the robotic actuator has been controlled to re-position to select another well (cf. FIG. 2; X-Y-movement), at box 3040.

Thus, by executing multiple iterations 3090, it is possible that the processor controls the robotic actuator to dip the electrical sensor into multiple wells of the MWP.

Once all iteration 3090 have been completed, i.e., time sequences of data readouts have been required for all required wells, the method then commences at box 3030. Here, one or more properties associated with molecules are determined based on the time sequence(s) of data readouts for each iteration 3090.

As a general rule, techniques for determining properties of the analyte molecules such as, e.g., binding properties to target molecules based on the time sequences of data readouts are known in the art and these techniques can be used herein. As a general rule, the analyte molecules may be attached to a sensor surface, i.e., may be sensor-side molecules. The analyte molecules could also be included in a liquid, i.e., liquid-side molecules.

For instance, it would be possible to determine an absolute signal level of the electrical signal captured by the data readouts. This absolute signal level could be compared against a reference. Thereby, it would be possible to determine a concentration. For instance, larger absolute signal levels can correspond to higher concentrations.

Another option would be to determine a change rate of the signal level. The change rate could be indicative of a binding kinetics. For instance, larger change rates could be indicative of faster binding.

These are only some options and other options are possible.

According to various examples, it is possible that the at least one property is determined at box 3030 based on the timing of said controlling of the robotic actuator to dip the electrical sensor into one or more wells of the MWP.

For instance, if a start time (defined by the timing of executing box 3005) or a stop time (defined by the timing of executing box 3020) of dipping the electrical sensor into a given well is known, this timing can be used in order to discriminate between data readouts acquired by the electrical sensor while the electrical sensor is being dipped into the respective well or before and after the electrical sensor is being dipped into the respective well. In particular, for low signal-to-noise ratios, it can be helpful to be able to judge between data readouts associated with only noise (i.e., before and after the electrical sensor is being dipped into a respective well) and data readouts associated with the signal (i.e., while the electrical sensor is being dipped into a respective well). This is, in particular, helpful if compared to manual techniques where a liquid including molecules is being pipetted onto the sensitive surface. Here, due to the manual process and surface tension preventing immediate wetting of the sensitive region, it can sometimes be difficult to judge when the sensitive region comes into contact with the (analyte) molecules.

The various iterations 3090 can be associated with or define different phases of a measurement protocol. Some of these phases are summarized in TAB. 2 below.

TABLE 2
Phases of a measurement protocol. The different phases can be implemented using a
measurement script. The measurement script can include (e.g., parameterized) control
instructions for controlling the robotic actuator and/or the electrical sensor to
implement such phases by dipping the electrical sensor into the respective well.

| Phase | Example description |
|---|---|
| Calibration | In the calibration phase, the robotic actuator can be controlled to dip |
| phase | the electrical sensor into a particular well that is filled with a reference |
| | liquid. For instance, the reference liquid could be water or a buffer |
| | solution. The reference liquid may not include a certain type of |
| | molecules, e.g., may not include analyte molecules. |
| | The reference liquid can have predefined properties so that the |
| | electrical signal output by the electrical sensor and captured by the |
| | respective time sequence of data readouts can serve as a reference. |
| Dissociation | A reference liquid can be used to (re-)transition the electrical sensor |
| phase | into a predefined state. For instance, if the sensitive region of the |
| | electrical sensor has been functionalized using sensor-side |
| | molecules, it would be possible that during the dissociation phase the |
| | sensor-side molecules are released from the sensitive surface, e.g., |
| | to go into solution in the reference liquid. |
| | During the dissociation phase it would also be possible to release |
| | liquid-side molecules that have been attached to sensor-side |
| | molecules. |
| Association | In the association phase, the robotic actuator can be controlled to dip |
| phase | the electrical sensor - that may or may not be functionalized using |
| | sensor-side molecules, see preparation phase - into a particular well |
| | that is filled with an analyte liquid. The analyte liquid includes the |
| | liquid-side molecules of a certain type. Then, the electrical signal of |
| | the electrical sensor is captured by the respective time sequence of |
| | data readouts can be indicative of one or more properties of the |
| | sensor-side molecules and/or the liquid-side molecules. |
| | For instance, the liquid-side molecules may come into contact with |
| | the sensitive region. For instance, the sensitive region may have been |
| | functionalized using sensor-side molecules and the liquid-side |
| | molecules may chemically bind to the sensor-side molecules. |
| Preparation | In the preparation phase, the robotic actuator can be controlled to dip |
| phase | the electrical sensor into a particular well that is filled with a liquid |
| | including sensor-side molecules. Then, the electrical sensor can be |
| | functionalized when the sensor-side molecules bind to the sensitive |
| | region of the electrical sensor. Thus, the preparation phase may |
| | precede the association phase. |

For instance, a concrete implementation of a measurement protocol could be as follows: (i) Well 1: Calibration phase-buffer solution, 5 min-establish baseline; (ii) Well 2: preparation phase-sensor-side molecules 5 min-adsorb sensor-side molecules onto surface of sensitive region, while simultaneously tracking, e.g., using a further electrical sensor dipped into a well not including a liquid that includes the sensor-side molecules and by comparing an offset between respective electrical signals, that the surface is indeed coated; (iii) Well 3: Calibration phase-buffer solution, 5 min-Confirm that sensor-side molecules are stuck onto the surface and do not detach; (iv) Well 4—association phase, liquid-side molecule 5 min-Binding of liquid-side molecules to sensor-side molecules; (v) Well 5: Dissociation phase—buffer solution, 5 min—Monitor how quickly the liquid-side molecules detach from the sensor-side molecule.

According to various examples, it would be possible that the processor is configured to control the robotic actuator to dip the electrical sensor into the one or more wells in accordance with a predefined measurement script that implements the measurement protocol. I.e., it would be possible that the measurement script specifies a time sequence of wells into which the electrical sensor is being dipped in different iterations 3090 (e.g., depending on the content of each well), a time duration or generally timing of dipping an electrical sensor into the one or more wells, etc. Thus, the measurement script can include a time sequence of control instructions that specifies the movement of the robotic actuator. Accordingly, it would be possible that decision-making at box 3015 and/or decision-making at box 3025 is based on a predefined measurement script. Such predefined measurement script, accordingly, can specify in which wells of the MWP the electrical sensor is to be dipped and for how long, whether or not the electrical sensor is to be stirred in a given well, etc. The predefined measurement script can also define a duration of the dipping and/or a count of data readouts of the time sequence per well.

According the various examples, it would be possible that the predefined measurement script is parametrized based on at least one parameter. A value of the at least one parameter can be set based on at least one of the data readouts.

In other words, the measurement script can be interactive. Based on the data readouts of the measurement, certain properties of the remaining actions of the measurement can be adjusted. This is achieved by setting the value of the at least one parameter based on the at least one of the data readouts.

By implementing the measurement script in an (auto-)parametrized manner, it is, in particular, possible to avoid dead times. For instance, it would be possible to minimize a duration of phases of the measurement protocol (cf. TAB. 2) by monitoring a time evolution of the time sequence of data readouts in the respective phase. Throughput of samples can be increased.

A few examples of such parametrization of the measurement script will be explained below.

For instance, it would be possible that the measurement script defines the calibration phase or dissociation phase(cf. TAB. 2). The parameter of the parametrized measurement script that has an adjustable value could be the dwell time of the electrical sensor in the well filled with the reference liquid. The value of the dwell time than could be set based on a change rate of multiple data readouts while the electrical sensor is dipped into the respective well, and/or in absolute signal level of the data readouts while the electrical sensor is dipped into the respective well such techniques are based on the finding that-after lowering the electrical sensor into the well filled with the reference liquid—it can be desirable to achieve a steady-state. In the steady-state, a reference value may be obtained from the respective data readouts and may be used to determine at least one property at box 3030.

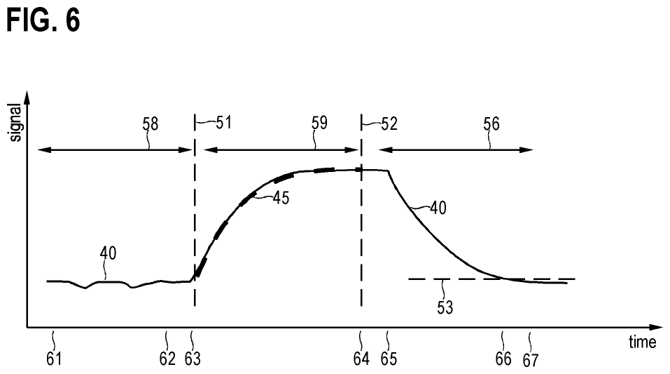

An example is illustrated in FIG. 6. FIG. 6 illustrates the data readouts 40—indicative of the signal level of the electrical signal of the electrical sensor 111—as a function of time. Here, the measurement scripts defines a calibration phase 58, between points in time 61 and 63, and a dissociation phase 56 between points in time 64 and 67.

For instance, in the calibration phase 58 between points in time 61 and 63, the signal level of the electrical signal of the electrical sensor 111 captured by the data readouts 40 is initially unsteady—i.e., has a significant change rate. Then, around point in time 62, the change rate drops below a certain threshold. This can trigger an end of the calibration phase 58 at point in time 63.

Then, in the dissociation phase 56 between points in time 64 and 67, the signal level of the electrical signal of the electrical sensor 111 captured by the data readouts 40 as initially significant amplitudes and then falls, at point in time 66, below a predefined threshold 53. This can trigger an end of the calibration phase 58 at point in time 67.

Next, another example of a parameter of a parametrized measurement script having an adjustable value that can be set based on the data readouts will be explained in connection with the association phase (cf. TAB. 2). FIG. 6 illustrates the association phase 59 between points in time 63 and 64.

For instance, the property to be determined could be a binding kinetics of liquid-side molecules to sensor-side molecules. In the association phase 59, the electrical sensor 111 can be dipped into a well that includes an analyte liquid including the liquid-side molecules. To determine the binding kinetics, a regression analysis can be used. Here, a predefined binding curve 45 is fitted to the signal levels of the data readouts 40 during the association phase 59. A parameter of the binding curve 45 can be proportional to a change rate of the signal level. Such parameter could be considered to determine the binding kinetics, in one example. Specifically, it would be possible that the regression analysis is performed in a certain time window or time gate. The value of this time gate can be set based on the timing of controlling the robotic actuator to dip the electrical sensor into the respective well filled with the analyte liquid. For instance, the time gate may start when the electrical sensor is lowered into the well (FIG. 3: box 3005) and may stop when the sensor is retrieved from the well (FIG. 3: box 3020). FIG. 6 illustrates a lower bound 51 of the time gate and an upper bound 52 of the time gate.

In FIG. 6, the calibration phase 58 and the association phase 59 and the dissociation phase 56 are executed in sequence. According to various examples, it would also be possible that the association phase in the calibration phase 58 or the dissociation phase 56 are executed in parallel—this may be helpful if, e.g., a signal level in a liquid without liquid-side molecules (i.e., the reference liquid of the calibration phase) is to be acquired as reference so that the absolute signal level in the association phase can be compared against the reference. More generally, it would be possible that a reference baseline is used when determining the at least one property. Such reference baseline could be obtained from a reference measurement that is implemented using a further electrical sensor. Here, it would be possible that different electrical sensors, e.g., electrical sensors 111, 112, are used to implement, in parallel, the calibration phase 58 in the association phase 59. For instance, the reference measurement could be implemented using the electrical sensor 112, while the electrical sensor 111 is used to acquire the data readouts that carry a signal indicative of the at least one property. For instance, the reference measurement could be used to quantify background noise or validate the data readouts acquired using the electrical sensor 111. Thus, according to various examples, it would be possible that the electrical sensor 111, as well as the electrical sensor 112 a lowered into the different wells 133 and 134 (cf. FIG. 1) that include the analyte liquid and the reference liquid, respectively. Where these electrical sensors 111, 112 are arranged at an offset matching an inter-well distance, they can be attached to the same robotic actuator 102. This increases the measurement throughput.

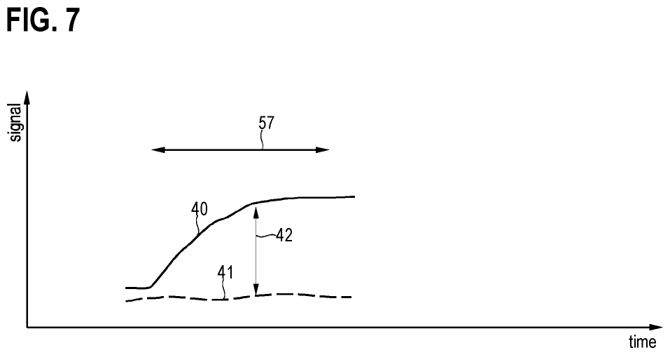

Another example would be to monitor the functionalization of the electrical sensor 111 during the preparation phase (cf. TAB. 2) using the further electrical sensor 112. This is illustrated in FIG. 7. FIG. 7 illustrates the data readouts 40—indicative of the signal level of the electrical signal of the electrical sensor 111—as a function of time. FIG. 7 also illustrates the further data readouts 41 (dashed line)—indicative of the signal level of a further electrical signal of the further electrical sensor 112—as a function of time during the preparation phase 57.

Here, it would be possible that the during the preparation phase 57 the robotic actuator 102 is controlled to dip the electrical sensor 111 into a well that includes a liquid including the sensor-side molecules while the electrical sensor 112 is dipped into another well that includes another liquid that does not include the sensor-side molecules. Thus, the sensitive region of the electrical sensor 111 is functionalized; while the sensitive region of the electrical sensor 112 is not functionalized. Then, an offset 42 between a signal level of the electrical signal of the electrical sensor 111 and captured by respective data readouts 40, and a further signal level of the further electrical signal of the further electrical sensor 112 and captured by respective further data readouts 41 can be tracked. For instance, a time dependency of this offset 42 could be tracked. For instance, where the change rate of this offset 42 falls below a certain threshold, it can be judged that the functionalization of the electrical sensor 111 has been completed. For instance, where the offset 42 is larger than a predefined threshold, can be judged that the functionalization of the electrical sensor 111 has been completed. Thus, the value of the dwell time can be set by tracking the offset 42. The functionalization can be validated by tracking the offset 42.

Although the invention has been shown and described with respect to certain preferred embodiments, equivalents and modifications will occur to others skilled in the art upon the reading and understanding of the specification. The present invention includes all such equivalents and modifications and is limited only by the scope of the appended claims.

1. A system, comprising:
a robotic actuator configured to engage an electrical sensor, the electrical sensor comprising a sensitive region that can be functionalized using first molecules of a first type,
a platform configured to retain a multi-well plate, and
at least one processor configured to control the robotic actuator to dip the electrical sensor into one or more wells of the multi-well plate, at least one of the one or more wells being filled with an analyte liquid comprising second molecules of a second type,
wherein the at least one processor is configured to control the electrical sensor to acquire one or more time sequences of data readouts when the electrical sensor is dipped into each one of the at least one of the one or more wells,
wherein the at least one processor is configured to determine at least one property of at least one of the first molecules or the second molecules based on at least some of the data readouts of the one or more time sequences of data readouts.

2. The system of claim 1,
wherein the at least one processor is configured to determine the at least one property based on a timing of said controlling of the robotic actuator to dip the electrical sensor into the one or more wells of the multi-well plate.

3. The system of claim 1,
wherein the at least one processor is configured to control the robotic actuator and the electrical sensor in accordance with a predefined measurement script,
wherein the predefined measurement script is parameterized based on at least one parameter, a value of the at least one parameter being set based on at least one of the data readouts of the one or more time sequences of data readouts.

4. The system of claim 3,
wherein the predefined measurement script defines a calibration phase or dissociation phase,
wherein the at least one processor is configured to control the robotic actuator to dip the electrical sensor into a first one of the one or more wells of the multi-well plate filled with a reference liquid during the calibration phase or the dissociation phase,
wherein the at least one parameter of the predefined measurement script comprises a dwell time of the electrical sensor in the first one of the one or more wells,
wherein the value of the dwell time is set based on at least one of a change rate of multiple data readouts of the data readouts while the electrical sensor is dipped into the first one of the one or more wells, or an absolute signal level of the multiple data readouts while the electrical sensor is dipped into the first one of the one or more wells.

5. The system of claim 3,
wherein the predefined measurement script defines an association phase,
wherein the at least one processor is configured to control the robotic actuator to dip the electrical sensor into the at least one of the one or more wells of the multi-well plate filled with the analyte liquid during the association phase,
wherein the at least one property comprises a binding kinetics, the binding kinetics being determined using a regression analysis of a predefined binding curve to multiple data readouts of the data readouts of a time sequence of data readouts acquired during the association phase,
wherein the at least one parameter of the predefined measurement script comprises a time gate for the regression analysis,
wherein a value of the time gate is set based on a timing of said controlling of the robotic actuator to dip the electrical sensor into the at least one of the one or more wells of the multi-well plate filled with the analyte liquid.

6. The system of claim 1,
wherein the robotic actuator is configured to engage a further electrical sensor so that the electrical sensor and the further electrical sensor are arranged at an offset which corresponds to an offset between wells of the multi-well plate,
wherein the at least one processor is configured to control the further electrical sensor to acquire one or more further time sequences of further data readouts when the further electrical sensor is dipped into each one of one or more further wells of the multi-well plate,
wherein the at least one processor is configured to determine the at least one property using a reference baseline obtained from at least some of the further data readouts.

7. The system of claim 6,
wherein the at least one processor is configured to control the robotic actuator and the electrical sensor in accordance with a predefined measurement script,
wherein the predefined measurement script defines a preparation phase,
wherein the at least one processor is configured to control the robotic actuator to dip the electrical sensor into a second one of the one or more wells of the multi-well plate filled with a liquid comprising the first molecules during the preparation phase,
wherein the at least one processor is configured to not dip the further electrical sensor into any well of the multi-well plate filled with the liquid comprising the first molecules during the preparation phase.

8. The system of claim 1, any one of the preceding claims,
wherein the at least one processor is configured to control at least one of the robotic actuator or a motor attached to the platform to relative move the electrical sensor with respect to and within the at least one of the one or more wells when the electrical sensor is dipped into each one of the at least one of the one or more wells.

9. The system of claim 1,
wherein the at least one property comprises at least one of a binding kinetics of a binding between the first molecules and the second molecules, binding affinity of the binding between the first molecules and the second molecules, concentration of the second molecules in the analyte liquid, or a conformality structure of the second molecules.

10. A computer-implemented method, comprising:
controlling a robotic actuator that engages an electrical sensor that can be functionalized using first molecules of a first type to dip the electrical sensor into one or more wells of a multi-well plate, at least one of the one or more wells being filled with an analyte liquid comprising second molecules of a second type,
controlling the electrical sensor to acquire one or more time sequences of data readouts when the electrical sensor is dipped into each one of the one or more wells, and
determining at least one property of at least one of the first molecules or the second molecules based on at least some of the data readouts.
